(19) United States
(12) Patent Application Publication
(10) Pub. No.: US 2014/0248584 A1
(43) Pub. Date: Sep. 4, 2014

(54) FILLED AND UNFILLED ORGANICALLY MODIFIED, OPTIONALLY ORGANICALLY CROSS-LINKED SILICIC ACID (HETERO) POLYSILICATES AS DENTAL MATERIALS FOR CHAIRSIDE TREATMENT

(71) Applicant: Fraunhofer-Gesellschaft zur Foerderung der angewandten Forschung e.V., Muenchen (DE)

(72) Inventors: Herbert Wolter, Tauberbischofsheim (DE); Florian Hausler, Wurzburg (DE)

(21) Appl. No.: 14/350,750

(22) PCT Filed: Oct. 10, 2012
(86) PCT No.: PCT/EP2012/070042

§ 371 Date: Apr. 9, 2014

(30) Foreign Application Priority Data
Oct. 12, 2011 (DE) 102011054444.5
Feb. 10, 2012 (DE) 102012202005.5

(51) Int. Cl. A61K 6/093; A61K 6/083; A61C 13/00

(52) U.S. Cl. CPC A61K 6/093; A61C 13/0004; A61C 13/0022; A61K 6/083
USPC 433/202.1; 433/218

(57) ABSTRACT

The present invention relates to an organically modified silicic acid (hetero) polycondensate or a composite comprising an organically modified silicic acid (hetero) polycondensate and a filler material for use in a dental procedure, particularly in a dental medicine procedure comprising at least a part or all of the following steps:
(a) Transfer of said organically modified silicic acid (hetero) polycondensate or said composite in the form of a block and hardening of said block through organic cross-linking, preferably in a thermal and/or redox-induced manner,
(b) Intra-oral measuring of the area requiring treatment in the mouth of the patient or a model or impression of this area outside the patient's mouth with the aid of a 3D imaging device,
(c) Transfer of the produced imaging data to a computer unit,
(d) Production of a virtual design to be inserted in the mouth of the patient with the aid of said computer unit, then either
(e1) Grinding out a fully anatomical form of this design from a block made of hardened organically modified silicic acid (hetero) polycondensate or said composite, or
(e2) Grinding out a reduced form with respect to this design from a block made of hardened organically modified silicic acid (hetero) polycondensate, coating the reduced form with an outer layer comprised of a pasty material, e.g. an organic material, hardening of the coating through organic cross-linking, and grinding out the fully anatomical form,
(g) Potentially polishing the form, and
(h) Inserting the form into the mouth of the patient.

In an alternative of the invention, step (a) is not necessary and the steps (e1) and (e2) are replaced by the following steps:
(e) Grinding out a reduced form with respect to this design from a block of material, which is preferably made of hardened organically modified silicic acid (hetero) polycondensate or a respective composite,
(f) Coating the reduced form with an outer layer comprised of said organically modified silicic acid (hetero) polycondensate or said composite in liquid or paste form, hardening of said coating through organic cross-linking, and grinding out the fully anatomical form.

The present invention relates to materials based on organically modified silicic acid (hetero) polysilicates for use as crowns, inlays, onlays, 3-unit bridges, repair system for crowns, basic prosthesis materials, dentures, and veneer material.

For the long-term maintenance of severely damaged teeth, the previously prepared tooth stumps normally receive metal, veneer or all-ceramic crowns. Plastic-based full crowns are primarily used in dental technology as temporary replacements due to the fact that the materials can only withstand chewing pressure for a short period. Thus, they have not previously been worth considering for high-quality long-term care as a result of serious mechanical deficiencies. In contrast, “classic” tooth replacement materials are not possible for easily applicable (Chair-side treatment), high-quality long-term care due to serious deficiencies in the fabrication and processing technology. For this reason, a time-consuming, i.e. multiple costly treatments, the integration of a temporary crown, two traumas involving the tooth, two applications of local anesthetics, as well as an expensive fabrication of the final full crown by dental technicians in a dental laboratory have been necessary up to this point. Retouching or repairs are only possible to a certain degree or not at all without removing the tooth replacement, which would normally cause it to be destroyed and to have to be remade. Many orders have been sent abroad to lower-wage countries for years in order to keep preparation prices low.

However, the desire for digitalization (e.g. digital impressioning) using tooth replacement materials with the highest esthetics/functionality is increasingly growing. In 2007, the expenditure of statutory health insurance providers in Germany was over 10 billion euros with the trend rising. The resulting cost pressure on the healthcare system can only be confronted with inexpensive, though high-quality tooth replacement systems. This ambitious plan means providing an innovative tooth replacement material for crowing vital/non-vital tooth stumps and, thus, definitive, i.e. long-term care of severely damaged teeth in order to maintain them for a long period. The material must allow the dentist to produce and use the fully-anatomically formed crown or respective other components for restoration or cosmetic needs during a patient's visit (chair-side treatment) with the aid of a computer (particularly with the help of CAD/CAM procedures) after gathering the data (oral scanner). Multiple patient treatments involving a dental laboratory with the integration of a temporary tooth replacement, such as a crown, should be eliminated. The costs for this type of fabrication are far below that of a conventional metal, veneer or all-ceramic crown because the time consumption is significantly lower and dental technicians (dental laboratory) are not necessary. However, the need for the “chair-side” processing of the material basis and the final properties under oral conditions has not been able to be met with the classic material basis for crowns.

If CAD/CAM technology is intended to be used, the tooth replacement is primarily produced in three steps:
(a) The preparation is acquired by means of a 3D recorder, e.g. a digital camera, in the mouth or on an impression or a (plaster) model.
(b) The generated image data is then transferred to a computer unit. The restoration (inlay, onlay, partial crown, crown, bridge framework) is optionally designed with supporting design references from the tooth database.
(c) A milling machine grinds out the restoration from an industrially produced crown molding. In addition to checking the accuracy of the fit and potentially necessary corrections, in conclusion, potentially a final polishing or individualization of the tooth replacement may be necessary with coordinated veneer materials.

The target group of the chair-side concept is dentists or assisting personnel under their supervision. If possible, the tooth replacement should be made directly after tooth preparation and a final intra-oral measurement at the office and the patient should be cared for at the same visit. The factors of time and material expense are reduced through intra-oral digitalization.

The objective of the invention is providing materials for the aforementioned purposes, particularly for crowns, inlays, onlays, 3-unit bridges, repair systems for crowns, basic prosthesis materials, dentures, and veneer materials. In solving these problems, these materials can meet the following requirements due to the composition selected pursuant to the invention:
(a) Chair-side crowns, inlays, onlays, 3-unit bridges, temporary crowns:
(a1). The molding can be produced as a block (multi-layered design potentially made from variously colored materials, the translucency of which preferably increases from the inside outward, whereas each layer is separately pre-hardened and the entire block is then re-hardened, or one color—then compact design).
(a2). Disc-shaped, round blocks with a thickness of preferably approx. 1-2 cm, from which multiple crowns (e.g. 25 pieces) can be milled, are beneficial.
   Both variations (a1)) and (a2) should have a high breaking strength and normally a high fracture toughness, potentially with a progression of opaque (tooth neck) to translucent (cutting edge/occlusal surface). Hardening should be possible in a thermal and/or redox-induced manner, potentially in combination with a thermal final hardening of all layers. The crown can be positioned in the layers in the multi-layered block via software to achieve an optimal esthetic result.
(a3). Moldings should be available as highly-filled single-color blocks that are additionally covered in specific embodiments with a pasty, translucent incisal material, which is applied, hardened, and subsequently re-milled (see EP 2 246 008 A2).
(a4). Highly-filled single-color blocks as a basic material, hardened in a thermal or redox-induced manner, should have a high breaking strength and potentially a high fracture toughness; the block can be milled as a reduced crown (milled significantly smaller than the anatomical form), a translucent incisal material can be applied manually in the deficit, and e.g. hardened with blue light, to which the final form should be milled, as provided for in application EP 2 246 008 A2. Prior to applying the incisal material, measures can potentially be taken for an improved bond of the components, such as roughening the surface of the reduced crown or applying a bonding agent. A high breaking strength can be realized through the use of various silanized and non-silanized filler materials in the nm and μm range as well as from combinations (see, e.g. Design Examples IV Example Set 3). The enamel layer can be realized in translucent form—this may occur through adaption of the refractive indices of the matrix and the filler material as well as through the incorporation of nano filler materials or special filler materials (see, e.g. Design Examples III Example Set 2). A high abrasion resistance can be achieved through the nano filler materials or through matrix systems with a high inorganic share. The breaking strength of the incisal material is less important with respect to the abrasion resistance because the basic material stabilizes the incisal material (see commercial veneer materials). The ability to polish the incisal material has to be ensured; there has to be bond strength between the base and incisal material.
   All prefabricated blocks pursuant to (a1) to (a4) can be further individualized with the aid of a light-hardening paint sets.
   One particular advantage of specific materials for crowns pursuant to the inventions according to (a1) to (a4) is the ability to perform repairs. In the case of a defect in the crown, e.g. chipping, it is possible for a dentist to repair it with a composite material capable of being hardened with light or redox-induced. A defective ceramic crown always has to be reproduced because it cannot be repaired intra-orally. Additional disadvantages of ceramic materials are its brittleness and complex retouching after the cutting process. Ceramic crowns have to be glaze fired after the cutting process to achieve a smooth surface and thus to prevent plaque deposits. This glaze firing is time-consuming; additional equipment (firing ovens) is necessary, for which ceramic materials do not represent genuine chair-side care. Composite-based chair-side crowns can be quickly polished by a dentist after the cutting process, through which the same surface quality is produced.
   For the temporary care, e.g. of the specified crowns, inexpensive filler materials can be used (see, e.g. Design Examples II Example Set 1). In these cases, the refractive index of the filler materials is frequently not aligned to the matrix. Thus, the esthetic is not optimal. However, in these cases this is not a condition because it only represents a transitional solution until the esthetical and high-quality tooth replacement has been produced.
(b) Repair systems for crowns
   A repair system (repair kit) should enable the repair of crowns, bridges, veneers, and the like. The composition and properties are comparable to those of the veneer material (see point (c)); however, suitable bonding agents must be present for various materials to ensure an optimal bond.
(c) Basic prosthesis materials, dentures, veneer material
   Basic prosthesis materials should be translucent, have a high breaking strength, and be easy to process. The invention can provide this as well. For example, norbornene-based matrices (output materials, e.g. silanes of a general formula F—see below) are suitable for this. Shrinkage should be as minimal as possible to ensure the form stability, which can be realized through the incorporation of filler materials. For instance, filler materials adapted to the refractive index as well as pearl and/or splinter polymerisates are possible. The matrix should be capable of being hardened in a redox-induced manner; mixed material should be capable of being cast to the extent possible. However, thermal hardening is possible as well; due to the individuality of prostheses and the resulting different material thicknesses, light-hardening may however be precluded.
   Requirements for dentures are: high translucency, high abrasion resistance, ability to be polished well. A good bond to the prosthesis base must be ensured, potentially by the fact that the area of the tooth neck is not as highly cross-linked. Any type of hardening should be possible. The invention also provides for this.
   Veneer material can be produced in a highly viscous and thixotropic manner. It can cover a broad variety of colors. The following applies for veneers on metal frames: bonding agents+opaque intermediate layer to cover the metal color, high abrasion resistance by nano filler materials and ability to be polished well. Veneers are modeled exclusively by hand (primarily for telescopic crowns on combination prostheses (combination of partial prostheses with crowns).

The objective of the invention is solved through the provisioning of an optionally filled silicic acid (hetero) polycondensate for use in “chair-side” applications, particularly as a repair material, basic prosthesis material, and veneer material, as well as the use of a filled, preferably thermally cross-linked silicic acid (hetero) polycondensate as a molding for “chair-side” crowns, inlays and onlays, and dentures, preferably in a surgical or therapeutic procedure to be conducted by a dentist. Particularly preferably, this occurs pursuant to one of the Claims 1 or 7 as well as one of the Claims dependent thereon.

According to the invention, the term used in the Claims “fully anatomical” as an attribute of a form means this form already has the final dimensions for insertion into the mouth of the patient or must only be minimally readjusted (e.g. subsequently cut), e.g. for the production of an optimal chew level. Likewise, according to the invention, the term “reduced” used there as an attribute of a form means that this form is dimensioned slightly too small. Both terms are standard terms in the field of the present invention.

The silicic acid (hetero) polycondensates used pursuant to the invention represent a common material basis for all aforementioned materials. Thus, they can be combined better, whereas properties such as esthetics, impact strength, breaking strength, modulus of elasticity, abrasion, and the like can be adjusted depending on the individual indication for a precise composition of the selected resin matrix, the type of filler material, and their shares to each other. As such, e.g. a modulus of elasticity can be randomly adjusted between, e.g. 1 GPa and 22 GPa, according to the requirements of the specific application and thus also the modulus of elasticity to be preferably achieved for the invention in the range of at least 4 GPa and in particular cases of at least 10 GPa. A similar situation applies for the breaking strength, which should favorably be at least at 80 MPa, in some embodiments at least at more than 90 MPa. Peak values of up to 150 MPa and even higher can be achieved.

The silicic acid (hetero) polycondensates used pursuant to the invention are mostly known from the state of the art. They all have a radical in common that is bonded to silicon via carbon, which normally bears at least one organically polymerizable group or one reactive ring. An organically polymerizable group presently means that this group is accessible to a polyreaction, for which reactive double bonds or rings transform into polymers (addition polymerization or chain-growth polymerization) under the influence of heat, light, ionizing radiation or redox-induced (e.g. with an initiator (peroxide or the like) and an activator (amine or the like)). During this polymerization, neither a separation of molecular components occurs nor a migration or rearrangement. Moreover, these groups should particularly preferably be accessible to a thiol-ene polyaddition when a thiol is added; even primary and secondary amines (particularly with at least two, though even three, four or more amino groups) should be able to be deposited. Alternatively, they can be accessible to a ROMP (ring opening metathesis polymerization). Examples for this are norbornene groups. The reactive double bond(s) of this group can be randomly selected, for example, a vinyl group or component of an allyl or styryl group. Preferably, it/they are a component of a double bond, which is accessible to a Michael addition, thus containing an activated methylene group as a result of the proximity to a carbonyl group. In turn, preferred among these are acrylic acid and methacrylic acid groups or derivatives. The organically polymerizable group normally contains at least two and preferably up to approx. 100 carbon atoms. It can be bonded to the carbon network of the Si—C bonded radical directly or via a random linkage group.

The term “(meth)acrylic . . . ” presently means that in each case it can be dealing with the respective acrylic or the respective methacrylic compound or a mixture of both. The present (meth)acrylic acid derivatives comprise the acids themselves, potentially in an activated form—esters, amides, thioesters, and the like.

The organically modified silicic acid polycondensates of the present invention may be exclusively silicon-based; however, instead they may have additional metal atoms in the inorganic framework as well, such as is known from the state of the art. These will be designated at present as silicic acid hetero polycondensates. The term “silicic acid (hetero) polycondensates” should comprise both variations. The condensates contain organic radicals bonded to silicon via carbon.

Examples for silicic acid hetero polycondensates usable pursuant to the invention, which are by no means limited, can be produced from the following silanes.

Silanes of a general formula (A):

{XₐR_{b}Si[R′(A)_{c}]_{(4-a-b)}}ₓB (A)

wherein said radicals have the following meaning:
X: hydroxy, alkoxy, acyloxy, alkylcarbonyl, alkoxycarbonyl or —NR″2;
R: alkyl, alkenyl, aryl, alkylaryl or arylalkyl;
R′: alkylene, arylene or alkylenarylene;
R″: hydrogen, alkyl or aryl;

### A: O, S, PR″, POR″ or NHC(O)O;

B: straight-chain or branched out organic radical that is derived from a compound with at least two C═C double bonds and 5 to 50 carbon atoms,
a: 1, 2 or 3;
b: 0, 1 or 2;
c: 0 or 1;
x: whole number, the maximum value of which corresponds to the number of double bonds in the compound B minus 1,

Such silanes and polycondensates produced therewith are revealed in DE 40 11 044 A1.

Silanes of a general formula (B):

B{A-(Z)_{d}—R¹(R²)—R′—SiXₐR_{b}}_{c} (B)

wherein said radicals and indices have the following meaning:

### A=O, S, NH or C(O)O;

B=straight-chain or branched out organic radical with at least one C═C double bond and 4 to 50 carbon atoms;
R=alkyl, alkenyl, aryl, alkylaryl or arylalkyl;
R′=alkylene, arylene, arylenalkylene or alkylenarylene with respectively 0 to 10 carbon atoms, wherein these radicals can be interrupted by oxygen and sulfur atoms or by amino groups;
R¹=nitrogen, alkylene, arylene or alkylenarylene with respectively 1 to 10 carbon atoms, wherein these radicals can be interrupted by oxygen or sulfur atoms or by amino groups;

### R²═H, OH or COOH;

X=hydrogen, hydroxy, alkoxy, acyloxy, alkylcarbonyl, alkoxycarbonyl or —NR″₂;
R″=alkyl or aryl;
Z═CO or CHR, with R equal to H, alkyl, aryl or alkylaryl;
a=1, 2 or 3;
b=0, 1 or 2;
c=1, 2 or 3
d=0 or 1

Such silanes and silicic acid polycondensates produced therewith are revealed in DE 44 16 857 C1.

Silanes of a general formula (C)

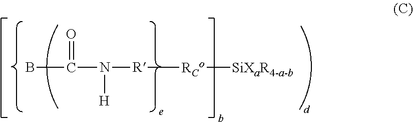

wherein said radicals and indices have the following meaning:
B=organic radical with at least one C═C double bond;
R=alkyl, alkenyl, aryl, alkylaryl or arylalkyl;
R^{o} and R′ respectively=alkylene, alkenylene, arylene, alkylenarylene or arylenalkylene;
X=hydroxy, alkoxy, acyloxy, alkylcarbonyl, alkoxycarbonyl or —NR″₂ with R″ equal to hydrogen, alkyl or aryl;
a=1, 2 or 3
b=1, 2 or 3, with a+b=2, 3 or 4;
c=0 or 1;
d=1, 2, 3, 4, 5, 6, 7, 8, 9 or 10;
e=1, 2 or 3 or 4; with e equal to 1 for c=0.

Said silanes of formula (C) and silicic acid polycondensates capable of being derived thereof are revealed in DE 199 10 895 A1. Silanes of a general formula (D):

{B′—Z′—R¹(B)—R—}ₐ(R)_{b}SiX_{4-a-b} (D)

wherein said radicals and indices have the following meaning:
R is an alkylene, arylene or alkylenarylene group, which can be interrupted by one or more oxygen or sulfur atoms or carboxyl or amino groups, or can carry such atoms/groups on its end facing away from the silicon atom;
R¹ is an alkylene, arylene or alkylenarylene group substituted by Z′, which can be interrupted by one or more oxygen or sulfur atoms or carboxyl or amino groups, or can carry such atoms/groups on one of its ends;
R′ is an alkyl, alkenyl, aryl, alkylaryl or arylalkyl group;
B and B′ can be equal or different; both radicals have the meaning of a straight-chain or branched organic group with at least one C═C double bond and at least two carbon atoms;
X is a group, which can enter a hydrolytic condensation reaction through the formation of Si—O—Si bridges (with the exception of hydrogen and halogen);
Z′ have the meaning —NH—C(O)O—, —NH—C(O)— or —CO(O)—, wherein both of the initially mentioned radicals are bonded to radical B′ by an NH group, while a carboxylate group can point in both directions;
a represents 1 or 2 and
b is 0 or 1;

Such silanes and polycondensates capable of being derived thereof are revealed in DE 103 49 766 A1.

Silanes of a general formula (E):

(XₐR_{b}Si)ₘ[—{B}—([O]ₒP[O]ₚR′_{c}Y_{d})ₙ]_{4ab} (E)

wherein said groups, radicals, and indices have the following meaning:
B is at least a double-bonded, straight-chain or branched group with at least one organically polymerizable radical and at least 3 carbon atoms,
X is a radical or OH capable of being hydrolyzed off a silicon atom (with the exception of hydrogen and halogen),
R and R′ are independent and potentially substituted alkyl, alkenyl, aryl, alkylaryl or arylalkyl,

### Y is OH or OR′,

a is 0, 1, 2 or 3,
b is 0, 1 or 2, wherein a+b together are 1, 2 or 3,
c is 0, 1 or 2,
d is 0, 1 or 2,
c+d together are 2,
m is at least 1, with the stipulation that m is not greater than 1 if a+b represents 1 or 2,
n is at least 1,
o is 0 or 1, and
p is 0 or 1,

Silanes of formula (E) and silicic acid polycondensates derived thereof are revealed in DE 101 32 654 A1.

Silanes of a general formula (F):

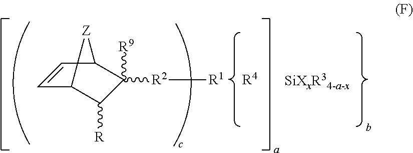

wherein said radicals and indices are equal or different and have the following meaning:
R is hydrogen, R²—R¹—R⁴—SiXₓR³₃₋ₓ, carboxyl, alkyl, alkenyl, aryl, alkylaryl or arylalkyl,
R¹ and R² are independent alkylene, arylene, arylenalkylene or arylenalkylene,
R³ is alkyl, alkenyl, aryl, alkylaryl or arylalkyl,
R⁴ is —(CHR⁶—CHR⁶)ₙ— with n=0 or 1, CHR⁶—CHR⁶—S—R⁵—, —C(O)—S—R⁵—, CHR⁶—CHR⁶—NR⁶—R⁵, —Y—C(S)—NH—R⁵, —S—R⁵, —Y—C(O)—NH—R⁵—, —C(O)—O—R⁵—, —Y—CO—C₂H₃(COOH)—R⁵—, —Y—CO—C₂H₃(OH)—R⁵— or —C(O)—NR⁶—R⁵,
R⁵ is alkylene, arylene, arylenalkylene or arylenalkylene,
R⁶ is hydrogen, alkyl or aryl with 1 to 10 carbon atoms,
R⁹ is hydrogen, alkyl, alkenyl, aryl, alkylaryl or arylalkyl,
X is hydroxy, alkoxy, acyloxy, alkylcarbonyl or alkoxycarbonyl;

### Y is —O—, —S— or NR⁶,

Z is —O— or —(CHR⁶)ₘ with m equal to 1 or 2;
a is 1, 2 or 3, with b=1 for a=2 or 3
b is 1, 2 or 3, with a=1 for b=2 or 3
c is a whole number from 1 to 6,
x is 1, 2 or 3 and
a+x are 2, 3 or 4.

These silanes and silicic acid polycondensates derived thereof are revealed in DE 196 27 198 A1.

Additional silicic acid (hetero) polycondensates usable pursuant to the invention comprise (meth)acrylic radicals and either sulfonate or sulfate groups that are respectively bonded directly or indirectly to a silicon atom via a non-substituted or substituted hydrocarbon group with a C—Si bond. These condensates can be produced, for example, from silanes of a formula (G),

R¹ₐR²_{b}SiZ_{4-a-b} (G)

wherein R¹ is a hydrolytically condensable radical, R² is substituted or non-substituted, straight-chained, branched or a cycle having alkyl, aryl, arylalkyl, alkylaryl or alkyl/arylalkyl or is a respective alkenyl, a carbon chain of may be interrupted in any event potentially by —O—, —S—, —NH—, —S(O)—, —C(O)NH—, —NHC(O)—, —C(O)O— —C(O)S, —NHC(O)NH— or C(O)NHC(O) groups, which may potentially point in both possible directions, Z is a radical, in which at least one (meth)acrylic group and at least either one sulfonate or one sulfate group is bonded directly or indirectly to a silicon atom via a non-substituted or substituted hydrocarbon group with a C—Si bond, a is 1, 2 or 3, b is 0, 1 or 2, and a+b together are 2 or 3. Specific silanes of said formula (G) have a following formula (G′)

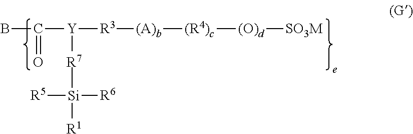

wherein
R³ is an alkylene, which is non-substituted or substituted with a functional group, straight-chained, branched, or having at least one cycle,
A represents a linkage group,
R⁴ represents an alkylene, which is potentially interrupted by O, S, NH or NR⁸ and/or potentially functionally substituted,
M is hydrogen or a monovalent metal cation or a respective share of a polyvalent metal cation, preferably selected from Na, K, ½Ca, ½Mg and ammonium,
R⁵ and R⁶ independently have either the meaning of R¹ or are alkyl, aryl, arylalkyl, alkylaryl or alkyl/arylalkyl, which are substituted or non-substituted, straight-chained, branched, or having at least one cycle; as an exception, however, may instead be a respective alkylene, arylalkylene or alkylenaryl,
R⁷ is a hydrocarbon group bonded to a silicon atom via a carbon atom, as was described further above,
R⁸ is C₁-C₆-alkyl or (meth)acrylic,
B is vinyl, 2-allyl or, in the case of e>1, an organic radical having e vinyl groups, which are respectively bonded to a group within the curly brackets
Y is a nitrogen atom, —O—CH═, —S—CH═ or —NH—CH═, wherein said oxygen atom, sulfur atom or said NH group has a bond to the neighboring C(O) group,
b=0 or 1
c=0 or 1
with the stipulation that, for the combination of Y equal to a nitrogen atom, b=0 and c=0 of said radical R³, as well as the stipulation that, for the combination of Y equal to a nitrogen atom, b=0 and c=1 of said radical R⁴ represents an alkylene interrupted by O, S, NH or NR⁸, and which is potentially functionally substituted,
d=0 or 1, and
e=1, 2 or 3.

Such materials are revealed in application DE 10 2011 050 672.1, which has not yet been published.

Additional silicic acid (hetero) polycondensates usable pursuant to the invention can be produced through a process, wherein a silane or siloxane having a radical bonded via a carbon atom to a silicon atom, which bears at least two functional groups, wherein a first functional group of the two is an unsaturated, organically polymerizable group and a second functional group of the two is selected from (a) additional unsaturated, organically polymerizable groups, (b) COOR⁸ or

—(O)_{b}P(O)(R⁵)₂ and (c) —OH, with R⁸ equal to R⁴ or M_{1/x}^{x+}, wherein M^{x+} is hydrogen or an x-fold positively charged metal cation, and b=0 or 1, is converted with a compound of a formula (H)

X—W—(Z)ₐ (H)

wherein X is SH, NH₂ or NHR⁴, Z is OH, a carboxylic acid radical —COOH or a salt or an ester of this radical or a silyl radical, W is a substituted or non-substituted hydrocarbon radical, the chain of which can be interrupted by —S—, —O—, —NH—, —NR⁴—,
—C(O)O—, —NHC(O)—, —C(O)NH—, —NHC(O)O—, —C(O)NHC(O)—, —NHC(O)NH—, —S(O)—, —C(S)O—, —C(S)NH—, —NHC(S)—, —NHC(S)O—, and a represents 1, 2, 3, 4 or a greater whole number, wherein R⁴ is a non-substituted or substituted hydrocarbon radical or OR⁶, R⁶ is hydrogen or a non-substituted or substituted hydrocarbon radical. The product of the first reaction is then converted in a second reaction with a compound (J)

Y—(W)k—(R1)b (J)

wherein Y is NCO, epoxy or—said radical or radicals Z is/are (a) hydroxy group(s) in the product of the first reaction—COA′, W is defined for compound (H) as above, R¹ is unsaturated, organically polymerizable group, A′ is hydroxy, a halogenide or —OC(O)R⁴ with R⁴ equal to a non-substituted or substituted hydrocarbon radical, k=0 or 1, wherein k=0 is only possible in the event that Y represents COA′ and b=1 or greater than 1.

This process is described in patent application DE 10 2011 053 865.8, which has not yet been published. If, in the process, silanes are used as output materials, a hydrolytic condensation will occur prior to, during or after any of the specified process steps.

The materials pursuant to the invention involve either masses comprised of organically polymerizable silicic acid (hetero) polycondensates, which, e.g. may be modified to achieve a higher degree of organic cross-linking—potentially with organic compounds (e.g. organic polymers) or other materials, or composites, i.e. polymerizable silicic acid (hetero) polycondensates potentially modified with organic compounds/materials, which are filled with filler materials. The filler materials can have any form and be especially particle-like and/or fibrous (particularly short fibers). The filler materials, for example, that are described in DE 196 43 781, DE 198 32 965, DE 100 184 05, DE 100 41 038, DE 10 2005 061 965, and DE 10 2005 018 305 are suitable. In necessary, a very high filler material content can be achieved.

The condensates are hardened via the above-presented polymerization reaction of groups containing double bonds and/or have rings. As an initiator for the thermal hardening, dibenzoyl peroxide (DBPO) is normally released in the respective resin system; other suitable hardeners are naturally likewise possible, such as those known from the state of the art. An organic cross-linking may occur through the addition of dimeric or oligomeric organic compounds to respective C═C double bonds, e.g. of thiols or amines with two or more thiol or amino groups. If thiols or amines in the deficit with regard to the available double bonds are used, remaining double bonds can be subsequently hardened.

For the incorporation of the filler, e.g. a three-roll mill or a planetary mixer can be used depending on the viscosity of the mixture. As such, the following methods were taken for the concrete design examples:
Three-roll mill: for lower viscosities, though in return very good deagglomeration of the filler materials; standard settings: 130 RPM
The deagglomeration of the filler materials is achieved through rollers running in opposite directions (speed and distance of the rollers in the μm range can be variably adjusted depending on the filler material size and type).
Planetary mixer: for higher viscosities, degassing is possible during the mixing process by applying negative pressure. The deagglomeration is achieved through two dough hooks running in opposite directions (speed variable).
There is the possibility of regulating the temperature of the three-roll mill and the planetary mixer->reducing the viscosity of the composite when incorporating
If different combinations of filler materials are use, the individual types of filler materials will be consecutively incorporated with the respective processes
Breaking strength and dry storage of the samples for modulus of elasticity/breaking strength in conformity with DIN 4049
Determination of conversion according to hardening via Raman spectroscopy
Measurements of translucency with a haze meter
2 mm thick, hardened sample discs
Wolfram halogen lamp produces light
Diffuse transmission of the light->translucency in %

The invention will be explained in further detail based on the following design examples:

### I. Resin Systems

Synthesis of Resin System A (from DE 44 16 857)

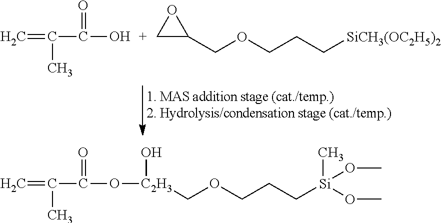

For receiving 125.0 g (0.503 mol) of 3-Glycidyloxypropyltrimethoxysilane, triphenylphosphine (as a cat.), BHT (as a stabilizer), and then 47.35 g (0.550 mol) of methacrylic acid are added drop-wise in a dry atmosphere and stirred at 80° C. (approx. 24 hrs.). The reaction can be followed by the decrease in the concentration of carboxylic acid via acid titration and the epoxy conversion can be followed via Raman spectroscopy/epoxy titration. The band of epoxy silane characteristic for the epoxy group appears in the Raman spectrum at 1256 cm⁻¹. The epoxy or carboxylic acid conversion is at ≧99% or ≧89% (→because 1:1.1 is a carboxylic acid surplus). After adding acetic ester (1000 ml/mol of silane) and H₂O for hydrolysis with HCl (as a cat.), it is stirred at 30° C. The progress of the hydrolysis is respectively followed via water titration. Processing occurs approximately after multiple days of stirring through repeated extraction with aqueous NaOH and with water and filtration via hydrophobized filters. A rotary evaporator is used first and then an oil pump vacuum is used for suctioning. This results in a liquid resin without the use of reactive thinners (monomers) having a very low viscosity of approx. 3-6 Pas at 25° C. (heavily dependent upon exact hydrolysis and processing conditions) and 0.00 mmol of CO₂H/g (no free carboxyl groups).

Synthesis of Resin System B (from DE 103 49 766.8)

### Basic Reaction Principle:

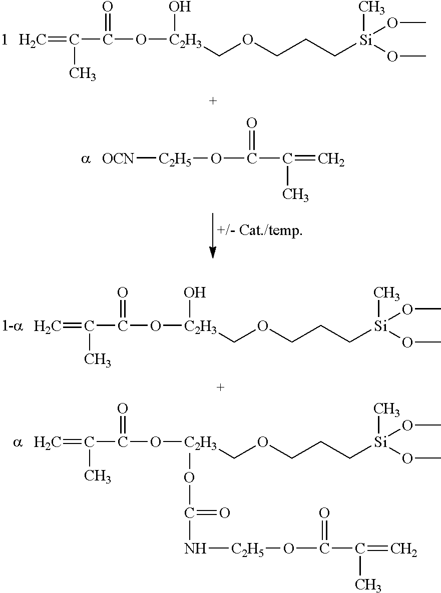

For receiving 53.1 g from Resin System A and 0.20 g of BHT, 21.7 g of methacrylic acid isocyanatoethyl ester are added drop-wise while stirring in a dry atmosphere at 30° C. and continually stirred at 30° C. The result is a liquid resin having a viscosity of approx. 22-28 Pas at 25° C. (heavily dependent upon precise synthesis and processing conditions, particularly the preliminary stages).

### Synthesis of Resin System C (DE 103 49 766.8)

### Basic Reaction Principle:

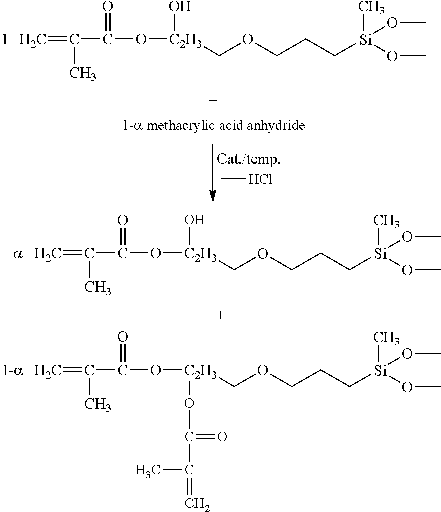

For receiving 174.2 g (0.65 mol) of Resin System A and 0.51 g of DABCO, 70.1 g of methacrylic acid anhydride are added drop-wise while stirring and continually stirred at 60° C. The reaction can be followed via NMR and by the decrease of anhydride bands via IR spectrum. Following the usual processing for separating the methacrylic acid released upon being added as well as the extraction of volatile components with the oil pump vacuum, a liquid resin emerges having a viscosity of approx. 2.1-2.4 Pa·s at 25° C. (heavily dependent upon precise synthesis and processing conditions, particularly the preliminary stages).

### Synthesis of Resin System D (DE 10 2011 053 865.8)

### Basic Reaction Principle:

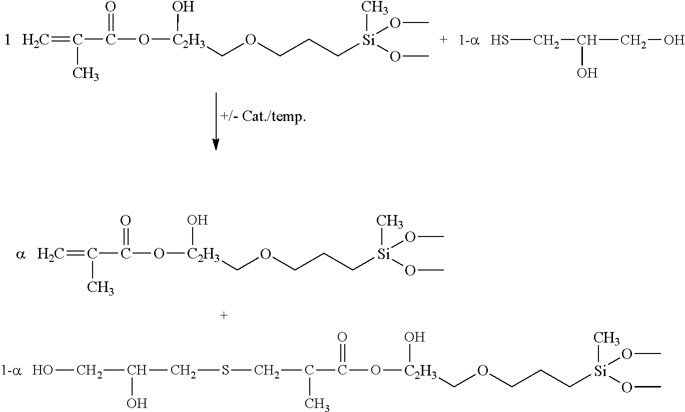

### Example of the 1^{st} Reaction:

For receiving 80.4 g (0.18 mol) of Resin System A and potentially 0.17 g of triethylamine, 8.96 g (0.276 mol) of 3-Mercaptopropane-1,2-diol are added drop-wise while stirring. The reaction can be followed via NMR and by the decrease of an HS band via IR spectrum. The band characteristic for said HS group appears in the Raman spectrum at 2566 cm⁻¹. The result is a liquid resin having a viscosity of approx. 16-18 Pas at 25° C. (depending on the exact synthesis and processing conditions of the preliminary stages). Additional processing is normally not necessary.

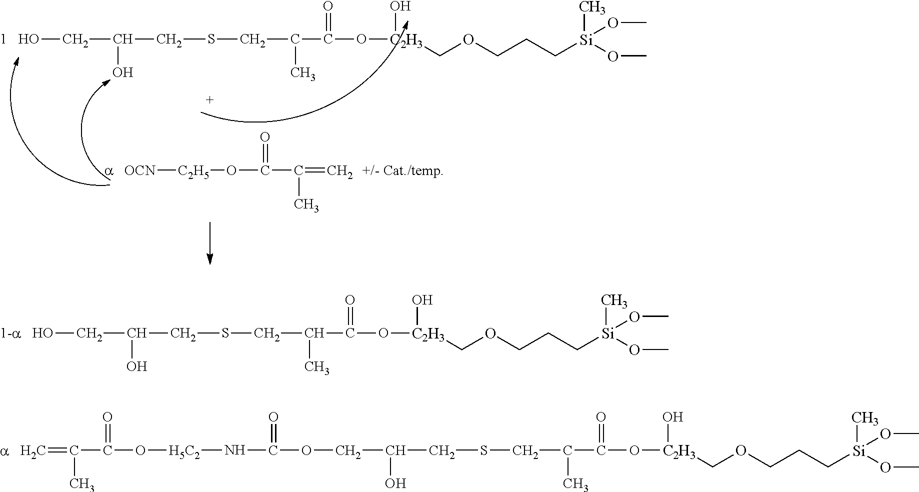

### Example of the 2^{nd} Reaction

For receiving 41.8 g from the example of the 1^{st} stage and potentially 0.12 g of BHT, 27.2 g (1.25 mol) of methacrylic acid isocyanatoethyl ester are added drop-wise while stirring in a dry atmosphere at 30° C. and continually stirred at 30° C. The result is a liquid resin.

### Synthesis of Resin System E (DE 44 16 857)

For receiving 129.2 g (0.52 mol) of 3-Glycidyloxypropyltrimethoxysilane, triphenylphosphine (as a cat.), BHT (as a stabilizer), and then 47.35 g (0.550 mol) of methacrylic acid are added drop-wise in a dry atmosphere and stirred at approx. 80° C. (approx. 24 h). The reaction can be followed by the decrease in the concentration of carboxylic acid via acid titration and the epoxy conversion can be followed via Raman spectroscopy/epoxy titration. The band of epoxy silane characteristic for the epoxy group appears in the Raman spectrum at 1256 cm⁻¹. After adding acetic ester (1000 ml/mol of silane) and H₂O for hydrolysis with HCl (as a cat.), it is stirred at 30° C. and 22.2 g (0.10 mol) of methacryloxy-methyltrimethoxysilane are added slowly drop-wise. The progress of the hydrolysis is respectively followed via water titration. Processing occurs after two days of stirring through repeated extraction with aqueous NaOH and with water and filtration via hydrophobized filters. A rotary evaporator is used first and then an oil pump vacuum is used for suctioning. This resulted in a liquid resin without the use of reactive thinners (monomers) having a viscosity of approx. 45.6 Pas at 25° C. (heavily depending upon the exact synthesis and processing conditions).

Resin System E differs from Resin System A by the fact that the output materials subjected to hydrolytic condensation additionally have methacryloxy-methyltrimethoxysilane, which leads to a stronger inorganic cross-linking of the resulting system.

### Synthesis of Resin System F (DE 10 2005 018 059)

For receiving 26.8 g (0.10 mol) of Resin System A, e.g. DBTL (as a cat.), BHT (as a stabilizer) are added drop-wise in a dry atmosphere (oxygen) at room temperature subsequently while stirring 1.68 g (0.01 mol) of hexamethylene-1,6-diisocyanate and stirred at 30° C. The reaction can be followed by the decrease of the OCN band via IR spectrum. The band characteristic for the OCN group appears in the IR spectrum at 2273 cm⁻¹. The result is a liquid resin having a viscosity of approx. 27 Pas at 25° C. (heavily dependent upon precise synthesis and processing conditions, particularly the preliminary stages).

The addition of hexamethylene-1,6-Diisocyanate leads to a stronger organic cross-linking of the resulting system.

### II Example Set 1: Composites for Temporary Crowns

Essential aspects are:
The use of a most inexpensive material, e.g. the use of Resin System A in combination with Quarz filler materials
The esthetic is less relevant in this case because the crowns are only used as a transition: the patient has to return to allow the work that will last for a long time to be finished and used.
The mechanical properties should be sufficient to be able to withstand the influences in an oral environment and the forces when chewing.
The breaking strength should exceed comparable PMMA-based materials (approx. MPa)

### EXAMPLE 1A

50% of Resin System A by weight+2% of DBPO
50% of Silmikron 810-10/1 filler material by weight (comprised of SiO₂ by nearly 99% by weight), primary particle size: 0.5 μm, non-silanized (company: Quarzwerke)
Incorporation of the filler: 3 passes in a three-roll mill
Thermal hardening for 4 hours at 100° C.; 1 day of dry storage at 40° C.
Breaking strength: 100±5 MPa
Modulus of elasticity: 4.5±0.11 GPa

### EXAMPLE 1 B

50% of Resin System A by weight+2% of DBPO
50% of Silbond 960-943 MST filler material by weight (comprised of SiO₂ by nearly 99% by weight), primary particle size: 1.2 μm, silanized (company: Quarzwerke)
Incorporation of the filler: 3 passes in a three-roll mill
Thermal hardening for 4 hours at 100° C., 1 day of dry storage at 40° C.
Breaking strength: 101±11 MPa
Modulus of elasticity: 5.1±0.08 GPa

### EXAMPLE 1C

50% of Resin System A by weight+2% of DBPO
50% of Silbond FW 600 MST filler material by weight (comprised of SiO₂ by nearly 99% by weight), primary particle size: 4 μm, silanized (company: Quarzwerke)
Incorporation of the filler: 3 passes in a three-roll mill
Thermal hardening for 4 hours at 100° C., 1 day of dry storage at 40° C.
Breaking strength: 113±8 MPa
Modulus of elasticity: 5.40±0.05 GPa

### EXAMPLE 1D

40% of Resin System A by weight+2% of DBPO
60% of Silbond FW 600 MST by weight (comprised of SiO₂ by nearly 99% by weight), primary particle size: 4 μm, silanized (company: Quarzwerke)
Incorporation of the filler: 2× three-roll mill
Thermal hardening for 4 hours at 100° C., 1 day of dry storage at 40° C.
Breaking strength: 126±10 MPa
Modulus of elasticity: 7.3±0.11 GPa

### EXAMPLE 1E

40% of Resin System A by weight+2% of DBPO
60% of a filler material mixture by weight (company: Quarzwerke)
Silmikron 810-10/1 (comprised of SiO₂ by nearly 99% by weight), primary particle size: 0.5 μm, non-silanized (2 passes in a three-roll mill)
Silbond FW 600 MST (comprised of SiO₂ by nearly 99% by weight), primary particle size: 4 μm, silanized (2×15 min. in a planetary mixer)
   Thermal hardening for 4 hours at 100° C., 1 day of dry storage at 40° C.
   Breaking strength: 131±14 MPa
   Modulus of elasticity: 7.0±0.14 GPa

### EXAMPLE 1F

40% of Resin System A by weight+2% of DBPO
60% of Silmikron 810-10/1 filler material by weight (comprised of SiO₂ by nearly 99% by weight), primary particle size: 0.5 μm non-silanized (company: Quarzwerke)
Incorporation of the filler: 3 passes in a three-roll mill
Thermal hardening for 4 hours at 100° C., 1 day of dry storage at 40° C.
Breaking strength: 148±8 MPa
Modulus of elasticity: 6.4±0.17 GPa

### III Example Set 2: Composites for Incisal Material (Highly Esthetic)

### Essential Aspects

High degree of translucence
High degree of hardness
Minimal abrasion
Can be polished well

### EXAMPLE 2A

71.4% of Resin System A by weight+2% of DBPO
28.6% of Silmikron 810-10/1 by weight (comprised of SiO₂ by nearly 99% by weight), primary particle size: 0.5 μm, non-silanized (company: Quarzwerke)
Incorporation of the filler: 1 pass in a three-roll mill
Thermal hardening for 4 hours at 100° C., 1 day of dry storage at 40° C.

| Breaking strength | Modulus of elasticity | Translucence | C═C reaction |
|---|---|---|---|
| [MPa] | [GPa] | [%] | [%] |
| 98 ± 8 | 3.1 ± 0.09 | 42 | 93 |

### EXAMPLE 2B

71.4% of Resin System A by weight+2% of DBPO
28.6% of Trisopor 4000 filler material by weight, non-silanized, porous glass, (267 nm pore size), amorphous, at least 90% of SiO₂ (company: VitraBio)
Incorporation of the filler: 3 passes in a three-roll mill
Thermal hardening for 4 hours at 100° C., 1 day dry at 40° C.

| Breakage strength | Modulus of elasticity | Translucence | C═C reaction |
|---|---|---|---|
| [MPa] | [GPa] | [%] | [%] |
| 111 ± 7 | 3.5 ± 0.07 | 41 | 98 |

### EXAMPLE 2C

71.4% of Resin System A by weight+2% of DBPO
28.6% of Ultrafine filler material by weight, primary particle size: 0.40 μm, silanized (55% of SiO₂ by weight,
25% of BaO by weight, 10% of B₂O₃ by weight, 10% of Al₂O₃ by weight) (Schott glass GM 27884)
Incorporation of the filler: 1 pass in a three-roll mill
Thermal hardening for 4 hours at 100° C., 1 day of dry storage at 40° C.

| Breakage strength | Modulus of elasticity | Translucence | C═C reaction |
|---|---|---|---|
| [MPa] | [GPa] | [%] | [%] |
| 100 ± 5 | 2.9 ± 0.05 | 53 | 94 |

### EXAMPLE 2D

71.4% of Resin System A by weight+2% of DBPO
28.6% Trisopor 400 filler material by weight, non-silanized, porous glass (40 nm pore size), amorphous, mind. 90% SiO₂) (company: VitraBio)
Incorporation of the filler: 2 passes in a three-roll mill
Thermal hardening for 4 hours at 100° C., 1 day of dry storage at 40° C.

| Breakage strength | Modulus of elasticity | Translucence | C═C reaction |
|---|---|---|---|
| [MPa] | [GPa] | [%] | [%] |
| 104 ± 7 | 3.6 ± 0.06 | 76 | 95 |

### EXAMPLE 2E

50% of Resin System A by weight+2% of DBPO
50% of nanofine filler material by weight, primary particle size: 0.18 μm, silanized (55% of SiO₂ by weight,
25% of BaO by weight, 10% of B₂O₃ by weight, 10% of Al₂O₃ by weight) (Schott glass GM 27884)
Incorporation of the filler: 1 pass in a three-roll mill, subsequently: vacuum process
Thermal hardening for 4 hours at 100° C., 1 day of dry storage at 40° C.

| Breakage strength | Modulus of elasticity | Translucence | C═C reaction |
|---|---|---|---|
| [MPa] | [GPa] | [%] | [%] |
| 127 ± 7 | 4.4 ± 0.04 | N.A. | N.A. |

### EXAMPLE 2F

75% of Resin System A by weight+2% of DBPO
25% of spray-dried nanoparticle filler material by weight, primary particle size: 70 nm, non-silanized (produced pursuant to DE 10 2005 061965). Incorporation of the filler: 1 pass in a three-roll mill
Thermal hardening for 4 hours at 100° C., 1 day of dry storage at 40° C.

| Breakage strength | Modulus of elasticity | Translucence | C═C reaction |
|---|---|---|---|
| [MPa] | [GPa] | [%] | [%] |
| 80 ± 3 | 2.4 ± 0.05 | N.A. | N.A. |

### EXAMPLE 2G

30% of Resin System E by weight+2% of DBPO
70% of a filler material mixture by weight, silanized (65% of SiO₂ by weight, 15% of B₂O₃ by weight, <5% of Al₂O₃ by weight, 5% of K₂O by weight, 10% of Cs₂O₃ by weight, 5% of La₂O₃ by weight, <5% of ZrO₂ by weight) (Schott glass G018-307), comprised of:
25% ultrafine, primary particle size: 0.7 μm (3 passes in a three-roll mill)
75% K5, primary particle size: 5.0 μm (2×15 min. in a planetary mixer, 40 RPM, 1×15 min.
with 0.8 bar of negative pressure (degassing)
   Thermal hardening for 4 hours at 100° C., 1 day of dry storage at 40° C.
   Breaking strength: 129±9 MPa
   Modulus of elasticity: 10.2±0.14 GPa

### EXAMPLE 2H

30% of Resin System E by weight+1% of Lucirin TPO
70% of a filler material mixture by weight, silanized (65% of SiO₂ by weight, 15% of B₂O₃ by weight, <5% of Al₂O₃ by weight, 5% of K₂O by weight, 10% of Cs₂O₃ by weight, 5% of La₂O₃ by weight, <5% of ZrO₂ by weight) (Schott glass G018-307), comprised of:
25% ultrafine, primary particle size: 0.7 μm (3 passes in a three-roll mill)
75% K5, primary particle size: 5.0 μm (2×15 min. in a planetary mixer, 40 RPM, 1×15 min.
with 0.8 bar of negative pressure (degassing)
   Photo-initiated hardening for 100 seconds on both sides, 1.5 days of dry storage at 40° C.
   Breaking strength: 123±9 MPa
   Modulus of elasticity: 8.9±0.44 GPa

### EXAMPLE 2I

30% of Resin System F by weight+1% of Lucirin TPO
70% of a filler material mixture by weight, silanized (65% of SiO₂ by weight, 15% of B₂O₃ by weight, <5% of Al₂O₃ by weight, 5% of K₂O by weight, 10% of Cs₂O₃ by weight, 5% of La₂O₃ by weight, <5% of ZrO₂ by weight) (Schott glass G018-307), comprised of:
25% ultrafine, primary particle size: 0.7 μm (3 passes in a three-roll mill)
75% K5, primary particle size: 5.0 μm (2×15 min. in a planetary mixer, 40 RPM, 1×15 min.
with 0.8 bar of negative pressure (degassing)
   Photo-initiated hardening for 100 seconds on both sides, 1.5 days of dry storage at 40° C.
   Breaking strength: 130±6 MPa
   Modulus of elasticity: 8.6±0.43 GPa

### EXAMPLE 2J

30% of Resin System B by weight+1% of Lucirin TPO
70% of a filler material mixture by weight, silanized (65% of SiO₂ by weight, 15% of B₂O₃ by weight, <5% of Al₂O₃ by weight, 5% of K₂O by weight, 10% of Cs₂O₃ by weight, 5% of La₂O₃ by weight, <5% of ZrO₂ by weight) (Schott glass G018-307), comprised of:
25% ultrafine, primary particle size: 0.7 μm (3 passes in a three-roll mill, temperature control 60° C.)
75% K5, primary particle size: 5.0 μm (2×15 min. in a planetary mixer, 40 RPM, 1×15 min.
with 0.8 bar of negative pressure (degassing)
   Photo-initiated hardening for 100 seconds on both sides, 1.5 days of dry storage at 40° C.
   Breaking strength: 142±10 MPa
   Modulus of elasticity: 9.2±0.38 GPa

### IV Example Set 3: Composites for Permanent Crowns/Basic Materials

### Essential Aspects

High breakage strength and modulus of elasticity
High level of fracture toughness
High level of hardness
Minimal abrasion
Comparable all-ceramic materials (feldspar ceramic) have a breaking strength of 113±10 MPa. These values should be exceeded pursuant to the invention.

### EXAMPLE 3A

15% of Resin System A by weight+1.5% of DBPO
85% of a filler material mixture by weight, silanized (55% of SiO₂ by weight, 25% of BaO by weight, 10% of B₂O₃ by weight, 10% of Al₂O₃ by (Schott glass GM 27884), comprised of:
18% nanofine, primary particle size: 0.18 μm (1 pass in a three-roll mill)
14% ultrafine, primary particle size: 0.40 μm (1 pass in a three-roll mill)
68% K6, primary particle size: 3.0 μm (2×15 min. in a planetary mixer, 20 RPM)
   Thermal hardening for 4 hours at 100° C., 1 day of dry storage at 40° C.
   Breaking strength: 167±15 MPa
   Modulus of elasticity: 13.8±0.66 GPa
   Vickers hardness: 100 HV 0.5; 30 s

### EXAMPLE 3B

12.5% of Resin System C by weight+1.5% of DBPO
One particularity of Resin System C is its relatively low viscosity—as a result, a higher filler material content is possible.
87.5% of a filler material mixture by weight, silanized (55% of SiO₂ by weight, 25% of BaO by weight, 10% of B₂O₃ by weight, 10% of Al₂O₃ by weight) (Schott glass GM 27884), comprised of
18% nanofine, primary particle size: 0.18 μm (1 pass in a three-roll mill)
14% ultrafine, primary particle size: 0.40 μm (2 passes in a three-roll mill)
68% K6, primary particle size: 3.0 μm (2×15 min. in a planetary mixer, 20 RPM, temperature control 60° C.)
Thermal hardening according to temperature program I (depicted in FIG. 2), 1 day of dry storage at 40° C.
Breaking strength: 166±12 MPa
Modulus of elasticity: 13.8±0.65 GPa
Vicker hardness: 95 HV 0.5; 30 seconds

### EXAMPLE 3C

30% of Resin System B by weight+2% of DBPO
70% of a filler material mixture by weight, silanized (50% of SiO₂ by weight, 1% of BaO by weight, 20% of SrO by weight, 15% of B₂O₃ by weight, 15% of Al₂O₃ by weight) (Schott glass GM 32087); from that
66.67% ultrafine, primary particle size: 0.70 μm (2 passes in a three-roll mill, temperature control 60° C.)
33.33% K5, primary particle size: 5.0 μm (2 passes in a three-roll mill, temperature control 60° C.)
   Thermal hardening for 4 hours at 100° C., 1 day of dry storage at 40° C.
   Breaking strength: 150±8 MPa
   Modulus of elasticity: 7.7±0.35 GPa

### EXAMPLE 3D

16% of Resin System B by weight+3% of DBPO
84% of a filler material mixture by weight, silanized (55% of SiO₂ by weight, 25% of BaO by weight, 10% of B₂O₃ by weight, 10% of Al₂O₃ by weight) (Schott glass GM 27884); from that
18% nanofine, primary particle size: 0.18 μm (1 pass in a three-roll mill, temperature control 60° C.)
14% ultrafine, primary particle size: 0.40 μm (1 pass in a three-roll mill, temperature control 60° C.)
68% K6, primary particle size: 3.0 μm (5×15 min. in a planetary mixer, 20 RPM, temperature control 60° C., 1×15 min. with 0.2 bar of negative pressure (degassing)
   Thermal hardening for 4 hours at 100° C., 1 day of dry storage at 40° C.
   Breaking strength: 162±16 MPa
   Modulus of elasticity: 16.5±0.62 GPa

### EXAMPLE 3E

16% of Resin System B by weight+2.5% of DBPO
84% of a filler material mixture by weight, silanized (55% of SiO₂ by weight, 25% of BaO by weight, 10% of B₂O₃ by weight, 10% of Al₂O₃ by weight) (Schott glass GM 27884); from that
18% nanofine, primary particle size: 0.18 μm (1 pass in a three-roll mill, temperature control 60° C.)
14% ultrafine, primary particle size: 0.40 μm (1 pass in a three-roll mill, temperature control 60° C.)
68% K6, primary particle size: 3.0 μm (2×15 min. in a planetary mixer, 50 RPM, temperature control 60° C.)
   Thermal hardening for 4 hours at 100° C., 1 day of dry storage at 40° C.
   Breaking strength: 166±10 MPa
   Modulus of elasticity: 16.2±0.68 GPa

### EXAMPLE 3F

16% of Resin System B by weight+2% of DBPO
84% of a filler material mixture by weight, silanized (55% of SiO₂ by weight, 25% of BaO by weight, 10% of B₂O₃ by weight, 10% of Al₂O₃ by weight) (Schott glass GM 27884); from that
18% nanofine, primary particle size: 0.18 μm (1 pass in a three-roll mill, temperature control 60° C.)
14% ultrafine, primary particle size: 0.40 μm (1 pass in a three-roll mill, temperature control 60° C.)
68% K6, primary particle size: 3.0 μm (5×15 min. in a planetary mixer, 20 RPM, temperature control 60° C., 1×15 min with 0.2 bar of negative pressure (degassing)
   Thermal hardening for 4 hours at 100° C., 1 day of dry storage at 40° C.
   186±10 MPa breaking strength
   16.1±0.47 GPa modulus of elasticity

### EXAMPLE 3G

30% of Resin System B by weight+2% of DBPO
70% of a filler material mixture by weight, silanized (65% of SiO₂ by weight, 15% of B₂O₃ by weight, <5% of Al₂O₃ by weight, 5% of K₂O by weight, 10% of Cs₂O₃ by weight, 5% of La₂O₃ by weight, <5% of ZrO₂ by weight) (Schott glass G018-307), comprised of:
25% ultrafine, primary particle size: 0.7 μm (3 passes in a three-roll mill)
75% K5, primary particle size: 5.0 μm (2×15 min. in a planetary mixer, 40 RPM, 1×15 min. with 0.8 bar of negative pressure (degassing)
   Thermal hardening for 4 hours at 100° C., 1 day of dry storage at 40° C.
   Breaking strength: 166±12 MPa
   Modulus of elasticity: 11.3±0.38 GPa

### EXAMPLE 3H

30% of Resin System F by weight+2% of DBPO
70% of a filler material mixture by weight, silanized (65% of SiO₂ by weight, 15% of B₂O₃ by weight, <5% of Al₂O₃ by weight, 5% of K₂O by weight, 10% of Cs₂O₃ by weight, 5% of La₂O₃ by weight, <5% of ZrO₂ by weight) (Schott glass G018-307), comprised of:
25% ultrafine, primary particle size: 0.7 μm (3 passes in a three-roll mill)
75% K5, primary particle size: 5.0 μm (2×15 min. in a planetary mixer, 40 RPM, 1×15 min.
with 0.8 bar of negative pressure (degassing)
   Thermal hardening for 4 hours at 100° C., 1 day of dry storage at 40° C.
   Breaking strength: 146±8 MPa
   Modulus of elasticity: 8.4±0.17 GPa

### EXAMPLE 3I

100% of Resin System D by weight+1.2% of camphor chinon+1.8% of DABE
Photo-initiated hardening for 100 seconds on both sides, 1.5 days of dry storage at 40° C.
Breaking strength: 130±4 MPa
Modulus of elasticity: 2.7±0.09 GPa

### EXAMPLE 3J

100% of Resin System D by weight+1% of Lucirin TPO
Photo-initiated hardening for 100 seconds on both sides, 1.5 days of dry storage at 40° C.
Breaking strength: 132±4 MPa
Modulus of elasticity: 2.7±0.11 GPa

### V Example Set 4: Single-Color Crowns

FIGS. 3a/3b and 4a/4b respectively show a cut and polished crown.

### EXAMPLE 4A

15% of Resin System A by weight (basic NHB-1840)+1.5% of DBPO
85% of a filler material mixture by weight, silanized (55% of SiO₂ by weight, 25% of BaO by weight, 10% of B₂O₃ by weight, 10% of Al₂O₃ by weight) (Schott glass GM 27884), comprised of:
18% nanofine, primary particle size: 0.18 μm (1 pass in a three-roll mill)
14% ultrafine, primary particle size: 0.40 μm (1 pass in a three-roll mill)
68% K6, primary particle size: 3.0 μm (2×15 min. in a planetary mixer, 20 RPM)
Thermal hardening for 3 hours at 100° C.

The obtained crowns are opaque with a certain translucency.

(Image of the crowns in FIG. 3a/3b)

### EXAMPLE 4B

30% of Resin System A by weight (basic NHB-2304)+2% of DBPO
70% of filler material by weight, non-silanized 0.7 μm (65% of SiO₂ by weight, 15% of B₂O₃ by weight, <5% of Al₂O₃ by weight, 5% of K₂O by weight, 10% of Cs₂O₃ by weight, 5% of La₂O₃ by weight, <5% of ZrO₂ by weight) (Schott glass GO 18-307)
Incorporation of the filler: Combination of the three-roll mill and planetary mixer
Thermal hardening for 4 hours at 100° C.

The obtained crowns are more translucent than those obtained pursuant to Example 4a.

(Image of the crowns in FIG. 4a/4b)

### EXAMPLE 5

As a composite for the production of the block, a composite made of Resin System A (p. 14 of the Description), 2% (dibenzoyl peroxide) of DBPO, and 72% of a filler material mixture by weight comprised of: 18% nanofine 180 (primary particle size 0.18 μm), 14% ultrafine 400 (primary particle size 0.40 μm), 68% K6 (primary particle size 3.0 μm), (GM 27884, company: Schott, composition: 55% of SiO₂ by weight, 25% of BaO by weight, 10% of B₂O₃ by weight, 10% of Al₂O₃ by weight) was used. The filler materials were incorporated with respectively 3 passes in the three-roll mill at 130 RPM. The crown composite was then polymerized (i.e. thermally hardened) into blocks for 4 hours at 100° C. A bracket for the CAD/CAM device was adhered to the blocks.

In the next step, a steel tooth stump (for the pressure test) was scanned with the aid of the CAD/CAM device. With the software, a fully anatomical crown was virtually modeled from each block and then milled down from the present crown composite blocks. Subsequently, the milled down crowns were blasted with corundum (250 μm grain size) with 2 bar of pressure at a distance of 1 cm from the surface, coated with a bonding agent, and thermally activated. The use of a bonding agent is not compulsory. In lieu of using a bonding agent, the crown may undergo, for example, a strong abrasion. A combination of both methods is possible.

For the second, more translucent layer, a pasty material was made from Resin System A, 1% of Lucirin TPO, and 70% of a filler material mixture by weight, comprised of: 25% ultrafine 400 (primary particle size 0.40 μm), 75% K5 (primary particle size 5.0 μm), (G018-307, company: Schott, composition: 65% of SiO₂ by weight, 15% of B₂O₃ by weight, <5% of Al₂O₃ by weight, 5% of K₂O by weight, 10% of Cs₂O₃ by weight, 5% of La₂O₃ by weight, <5% of ZrO₂ by weight). The filler materials were incorporated with respectively 3 passes in the three-roll mill at 130 RPM. The material was excessively applied to the previously milled down crowns and polymerized using a dental blue light halogen spotlight and thus hardened. The crowns prepared in this manner were then reinserted into the CAD/CAM device and milled fully anatomically.

### EXAMPLE 6

Example 5 was repeated with the modification that the crown composite was milled fully anatomically and no second, translucent coating was applied.

### COMPARISON EXAMPLE

With the processes specified for Examples 5 and 6, a fully anatomically milled crown was produced from commercially available feldspar ceramic.

### Results

Due to the 2-layer design, the crowns produced pursuant to Example 5 have a significantly higher esthetic than crowns pursuant to Example 6.

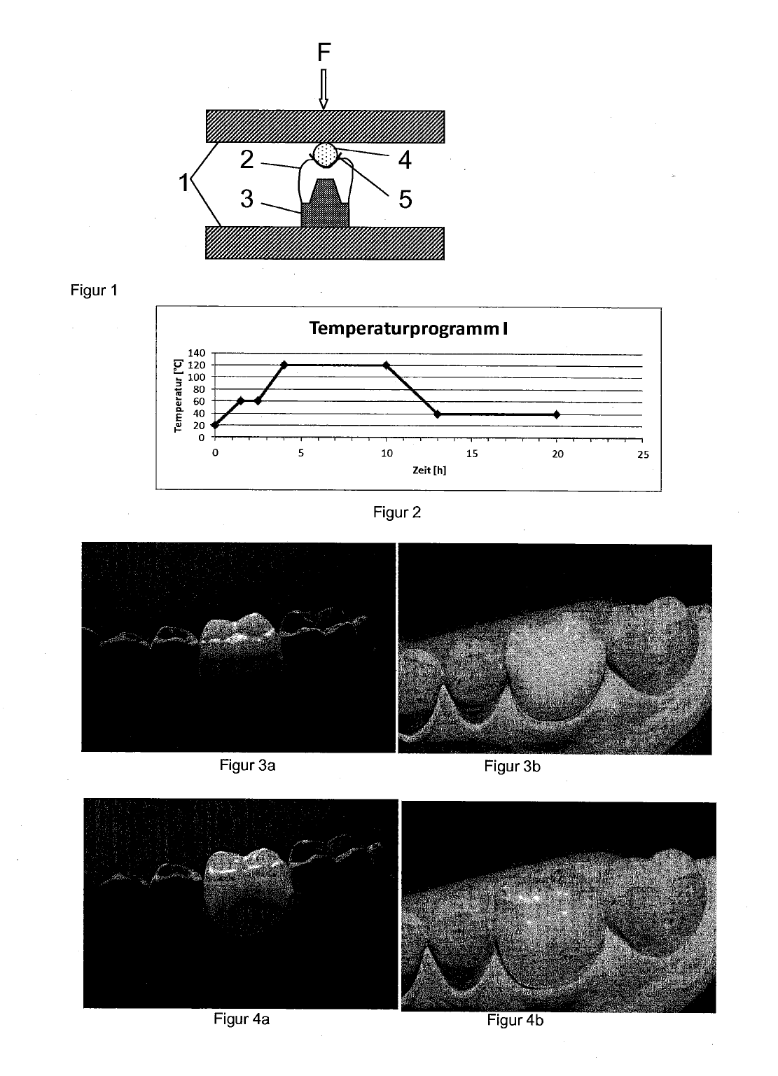

To determine the load capacity of the crowns produced according to the examples and the comparison example, a pressure test (see FIG. 1 for test configuration) was conducted. In this regard, the crowns were respectively applied over the steel stump. A hardened steel ball was placed onto the chewing surface. This transfers the force of the pressure plate to the crown. A 0.2 mm thick piece of aluminum foil between the crown and the steel ball served to minimize the concentration of stress during the test. Due to the fact that no defined surface fracture was expected with this test geometry, only the maximum test force was determined, at which the crowns failed.

For the crowns pursuant to Example 5, the maximum test force after being dry stored for 3 days at 40° C. was 2280±120 N. The crowns broke cohesively, i.e. a spalling of the outer, subsequently applied coating was not be detected.

The maximum test force of fully anatomical crowns pursuant to Example 6 force after being dry stored for 3 days at 40° C. was 2590 N±420 N.

The maximum test force of fully anatomical crowns made from commercially available feldspar ceramic was 2400 N±540 N directly after being produced.

### REFERENCE LIST

1 Pressure plates
2 Crown
3 Steel stump
4 Hardened steel balls
5 Aluminum foil

What is claimed is:

1. Dental medicine treatment, comprising a part or all of the following steps:
(a) Transforming an organically modified silicic acid (hetero) polycondensate or a composite comprising an organically modified silicic acid (hetero) polycondensate and a filler material into the form of a block and hardening of said block through organic cross-linking,
(b) Intra-oral measuring of the area requiring treatment in the mouth of the patient or of a model or a replica of this area outside the patient's mouth with the aid of a 3D imaging device,
(c) Transfer of the produced imaging data to a computer unit,
(d) Production of a virtual design to be inserted in the mouth of the patient with the aid of said computer unit, then
either
(e1) Grinding out a fully anatomical form of this design from a block made of hardened organically modified silicic acid (hetero) polycondensate or said composite,
or
(e2) Grinding out a reduced form with respect to this design from a block made of hardened organically modified silicic acid (hetero) polycondensate or said composite, coating the reduced form with an outer layer comprised of a pasty material selected from organic materials and inorganic-organic hybrid materials, hardening of the coating through organic cross-linking, and grinding out the fully anatomical form,
(g) Optionally polishing the form,
and
(h) Inserting the form into the mouth of the patient, optionally after checking the accuracy of the fit and making necessary corrections in this regard.

2. Dental medicine treatment according to claim 1, wherein said pasty material used in step (e2) is an organically modified silicic acid (hetero) polycondensate or a composite comprising an organically modified silicic acid (hetero) polycondensate and a filler material.

3. (canceled)

4. Dental medicine treatment according to claim 1, wherein multiple materials are used for said step (a), wherein said block is formed either from a core and one or more outer layers or from a multitude of layered coatings, and the color of said core and said layers changes from the inside outward and/or the translucency of said core and said layers increases from the inside outward.

5. (canceled)

6. Dental medicine treatment according to claim 1, wherein in step (e2) the surface of the ground out form is roughened and/or to which a bonding agent is applied before said reduced form is coated with an outer layer made of a pasty material.

7. Dental procedure, comprising at least a part or all of the following steps:
(a) Providing an organically modified silicic acid (hetero) polycondensate or a composite comprising an organically modified silicic acid (hetero) polycondensate and a filler material in a liquid or paste form,
(b) Intra-oral measuring of the area requiring treatment in the mouth of the patient or of a model or a replica of this area outside the patient's mouth with the aid of a 3D imaging device,
(c) Transfer of the produced imaging data to a computer unit,
(d) Production of a virtual design to be inserted in the mouth of the patient with the aid of said computer unit, then
(e) Grinding out a reduced form from a block of material with respect to the design,
(f) coating the reduced form with an outer layer comprised of said organically modified silicic acid (hetero) polycondensate or said composite in liquid or paste form, hardening the coating through organic cross-linking and grinding out the fully anatomical form,
(g) Optionally polishing the form,
(h) Inserting the form into the mouth of the patient, optionally after checking the accuracy of the fit and making necessary corrections.

8. Dental procedure according to claim 7, wherein in step (e) the surface of the ground out form is roughened and/or to which a bonding agent is applied before said reduced form is coated with said outer layer made of said organically modified silicic acid (hetero) polycondensate or the composite in liquid or paste form according to step (f).

9. Dental procedure according to claim 7, wherein the block of material used in step (e) consists of a hardened organically modified silicic acid (hetero) polycondensate or a composite comprising said organically modified silicic acid (hetero) polycondensate and a filler material.

10. (canceled)

11. Dental procedure according to claim 7, wherein said silicic acid (hetero) polycondensate has radicals bonded to silicon via carbon, which have at least one C═C double bond or a ring capable of a polymerization reaction.

12. Dental procedure according to claim 11, wherein at least two different radicals bonded to silicon via carbon are present.

13. Dental procedure according to claim 11, wherein at least part of said radicals has a group, which contains at least one C═C double bond accessible to a ROMP or to a thiol-ene addition.

14. Dental procedure according to claim 12, wherein said radicals bonded to silicon via carbon have at least two different groups having each at least one organically polymerizable C═C double bond.

15. Dental procedure according to claim 11, wherein said radicals bonded to silicon via carbon additionally have at least partially one reactive group selected from COOH, OH, a phosphorous containing group, particularly a phosphoric acid or ester group, a group containing sulfonic acid, and an amino group.

16-17. (canceled)

18. Dental procedure according to claim 1, wherein the procedure is chosen from the care of the patient by administering a crown, an inlay, an onlay, a prosthesis, a prosthesis tooth or a veneer material.

19. Dental procedure according to claim 7, wherein the procedure is a dental medical treatment.

20. Dental procedure according to claim 1, wherein said silicic acid (hetero) polycondensate has radicals bonded to silicon via carbon, which have at least one C═C double bond or a ring capable of a polymerization reaction.

21. Dental procedure according to claim 20, wherein at least two different radicals bonded to silicon via carbon are present.

22. Dental procedure according to claim 20, wherein at least one part of said radicals has a group, which contains at least one C═C double bond accessible to a ROMP or to a thiol-ene addition.

23. Dental procedure according to claim 21, wherein said radicals bonded to silicon via carbon have at least two different groups having each at least one organically polymerizable C═C double bond.

24. Dental procedure according to claim 20, wherein said radicals bonded to silicon via carbon additionally have at least partially one reactive group selected from COOH, OH, a phosphorous containing group, particularly a phosphoric acid or ester group, a group containing sulfonic acid, and an amino group.

25. Dental procedure according to claim 7, wherein the procedure is chosen from the care of the patient by administering a crown, an inlay, an onlay, a prosthesis, a prosthesis tooth or a veneer material.
